(19) United States
(12) Patent Application Publication
(10) Pub. No.: US 2003/0091520 A1
(43) Pub. Date: May 15, 2003

(54) Use of at least one polar additive in a cosmetic composition comprising a liquid fatty phase structured by at least one molecular organogelator, to impart a thixotropic character to the composition

(75) Inventors: Aude Livoreil, Paris (FR); Nawel Baghdadli, Massy (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/163,509

(22) Filed: Jun. 7, 2002

(30) Foreign Application Priority Data
Jun. 7, 2001 (FR) 01 07474

(51) Int. Cl. A61K 7/06

(52) U.S. Cl. 424/70.1

(57) ABSTRACT

The invention concerns the use of at least one polar additive having a polarity parameter δₐ equal to or greater than 7.0 (Jcm³)^{½}, in a cosmetic composition containing at least one apolar or weakly polar oil having a polarity parameter δₐ less than 7.0 (J/cm³)^{½}, structured by at least one molecular organo-gelator, to impart a thixotropic character to the composition.

The present invention relates to the use of a polar additive, in a cosmetic composition for the care and/or make-up of the skin and/or lips and/or hair or nails of the human body, comprising a liquid fatty phase containing at least one apolar or weakly polar oil, structured by at least one organo-gelator, to impart a thixotropic character to the composition.

These compositions, which may in particular be in solid form, particularly in sticks, lead after application to deposits which do not migrate, do not break up, are non-granulous and easy to spread.

By polar additive should be understood, in the context of the invention, a polar compound characterized by a polarity parameter δₐ equal to or greater than 7.0 (J/cm³)^{½}.

By apolar or weakly polar oil should be understood, in the context of the invention, an oil with a polarity parameter δₐ less than 7.0 (J/cm³)^{½}.

By polarity parameter should be understood, in the context of the invention, the average parameter δₐ describing the polarity of a molecule: the higher the value of δₐ, the higher the polarity of the molecule.

The average parameter δₐ is defined as a function of the Hansen solubility parameters δₚ and δₕ, according to the following relation:

δₐ=√(δₚ²+δₕ²)

The parameters δₚ and δₕ respectively characterize the Debye interaction forces between permanent dipoles and the capacity of a compound to form hydrogen bonds. These parameters are defined according to the space of the solubility parameters of HANSEN in the document J. Paint Technology 39, 195 (1967) “The Three Dimensional Solubility Parameter—Key to Paint Component Affinities”.

By liquid fatty phase should be understood, in the context of the invention, a fatty phase liquid at ambient temperature (25° C.) and at atmospheric pressure (76 mm of Hg), composed of one or more fatty bodies liquid at ambient temperature, also called oils, generally compatible between themselves.

By liquid fatty body should be understood, in the context of the invention, a nonaqueous liquid medium, immiscible in any proportion with water.

By organo-gelator should be understood, in the context of the invention, a molecular non-polymeric organic compound whose molecules are able to establish physical interactions between themselves leading to an auto-aggregation of the molecules with formation of a D3 supra-molecular network which is responsible for gelling the liquid fatty phase.

By gelation should be understood, in the context of the invention, a thickening of the medium leading to a gelatinous consistency and even to a rigid solid consistency, not flowing under its own weight.

By composition with a thixotropic character should be understood, in the context of the invention, a structured composition which becomes fluid when it is applied to the lips, skin, hair or nails, and which recovers all or part of its initial viscosity over time after application, at ambient temperature.

The use of molecular organo-gelators to structure the fatty phase of cosmetic and/or dermatological compositions containing a liquid fatty phase is known to a person skilled in the art.

However, such compositions have the disadvantage of showing a rheo-fluidizing behaviour.

By rheo-fluidizing behaviour should be understood, in the context of the invention, a composition which leads to a deposit which fluidizes during its application, but remains fluid and does not recover all or part of its initial viscosity, or only does so after a very long time (one month or more).

In addition, the use of molecular organo-gelators to structure the fatty phase leads after application to deposits which tend to break up, i.e. granulous deposits or deposits in the form of small fragments or scales.

A cosmetic composition showing a rheo-fluidizing behaviour may also lead to exudation of the fatty phase, especially in warm or humid areas. Such a composition may also encourage, after application to the skin or lips, the migration of this phase into the wrinkles or fine lines of the skin surrounding the lips and eyes, which is particularly undesirable for lipsticks or eye shadows.

In fact, a significant migration of the liquid fatty phase, in particular when it contains colorants, carries the colorant with it and leads to an unsightly effect around the lips and eyes, since it particularly accentuates the wrinkles and fine lines.

By migration should be understood, in the context of the invention, a spreading of the composition, and in particular of the colour, outside the initial position of the make-up.

There thus remains the need for compositions not showing the above disadvantages, and particularly compositions leading after application to deposits

which recover their initial viscosity in a very short time after application, in other words which solidify rapidly,

which do not migrate into the wrinkles or fine lines of the skin or lips,

which do not break up and are not granulous or heterogeneous, but which in contrast are homogenous, with a smooth and continuous texture,

which show ease of spreading and flowing properties.

In addition, the composition after shearing is smooth and creamy.

The applicant has discovered the surprising fact that the use of a polar additive, in a cosmetic composition comprising a liquid fatty phase, structured by at least one molecular organo-gelator, modifies the rheology of the composition, and in particular imparts a thixotropic character to it.

By thixotropic character of the composition should be understood, in the context of the invention, the property of the composition to fluidize under shearing, and in particular during its application to the skin, lips, hair or nails, and to return totally or in part to its original viscosity over time and at ambient temperature.

More particularly, the applicant has discovered that this polar additive only gives a thixotropic character to the composition if the fatty phase contains at least one apolar or weakly polar oil. The polar additive may be chosen from among the compounds considered as good donors or acceptors of H bonds, such as for example the fatty alcohols, the fatty acids, the diols, the esters, the pyrrolidones, the ketones or the sulfoxides.

A person skilled in the art knows the use of these polar additives as solvents or coupling agents in order to achieve the compatibilization of the compositions.

A document which may be cited for illustration is thus the publication “Method of Gelling Non-polar Oils with Dibenzylidene Sorbitol (DBS) through the Use of Co-solvents” by Dimitris E. Katsoulis and Janet M. Smith (Research Disclosure, August 1994, Dow Corning Corporation). This document describes the use of a method of gelling a silicone oil with dibenzylidene sorbitol (DBS) and a polar co-solvent, to produce gels with a high silicone level. The polar co-solvent is use to compatibilize the silicone oil to be gelled and the DBS and thus makes the gelation of the silicone oil by the DBS possible. The silicone gels obtained by this method may be used as vehicles for antiperspirant salts, pigments and sunscreens. They may be also used in the form of cosmetic gels or sticks, or in the form of antiperspirant or deodorant gels or sticks, or in the form of sunscreen gels or sticks.

The U.S. Pat. No. 5,403,580 discloses a method for gelling a silicone oil with a gelling agent derived from a steroid and a co-solvent used to dissolve the gelling agent in the silicone oil, the co-solvent being a volatile solvent such as chloroform (CHCl₃).

In addition, this U.S. Pat. No. 5,403,580 discloses a composition for an antiperspirant gel in the form of a stick, comprising an antiperspirant agent, a first gelling agent selected from the group of hydroxystearic acid and its derivatives (especially esters and amides), a second gelling agent selected from the group of N-acylaminoacid amide and its derivatives, and a polar co-solvent. The polar co-solvent increases the compatibility of the gelling agents and the silicone oil, which leads to a lower processing temperature and a higher proportion of oil in the stick. The co-solvent is selected from the fatty alcohols, the fatty acids, the ethers and the esters derived from the alcohols and acids, and in particular octyl dodecanol, undecyl pentadecanol, ethyl myristate, isopropyl palmitate, finsolv and diisopropyl sebacate.

Finally, the U.S. Pat. No. 5,958,386 discloses an antiperspirant composition comprising an antiperspirant agent, a volatile hydrocarbon oil, a gelling agent selected from the fatty acids, the esters of fatty acids, the amides of fatty acids and the derivatives of N-acylamino acid, and a coupling agent to compatibilize the gelling agent and the hydrocarbon oil and thus reduce its processing temperature. The coupling agents are preferably selected from among dimethiconol, octyidodecanol, hexyldecanol, octyldecanol, undecylpentadecanol, isopropyl myristate, diisopropyl adipate, ethanol and diethyl phthalate.

None of these documents concerns the use of a polar additive in a structured composition comprising an apolar or weakly polar oil and a molecular organo-gelator, to modify the rheology of thus composition by imparting to it a thixotropic character.

However, the international application WO-00015180-A1 describes a structured composition (thickened or gelled) comprising an apolar oil or wax, a compound to modify the rheology (gelling agent), preferably a polysaccharide, and a compound with H bonds to compatibilize the oil and the gelling agent. The gelling agent is not a molecular organo-gelator. In addition, this document teaches that the viscosity of the composition varies as a function of the proportion of coupling agent in the composition, but nothing is said about a profound modification of the rheology as a function of the proportion of coupling agent, and in particular the modification of the rheological behaviour of the structured composition towards a thixotropic behaviour when it contains a polar additive.

The object of the invention is thus the use of at least one polar additive having a polarity parameter δₐ equal to or greater than 7.0 (J/cm³)^{½}, in a cosmetic composition comprising a liquid fatty phase containing at least one apolar or weakly polar oil having a polarity parameter δₐ less than 7.0 (J/cm³)^{½}, structured by at least one molecular organo-gelator, to impart a thixotropic character to the composition.

In addition, the use of at least one polar additive such as defined above, in a cosmetic composition:

reduces or even eliminates the migration of the fatty phase into the wrinkles and fine lines of the skin or lips;

reduces the formation of ridges on the eyelids particularly when the composition is an eye shadow;

reduces or even eliminates the tendency of compositions structured by molecular organo-gelators to break up, and/or encourages the spreading of the composition onto the skin and/or the formation of a smooth, homogenous, non-granulous and continuous deposit on keratin-containing material.

favours the spreading of the composition onto the skin or lips.

The structuring of the liquid fatty phase may be modulated according to the nature of the molecular organo-gelator used, and the rheology of the composition may be perfectly adjusted by adapting the quantity of additive used.

Polar Additive

The polar additive according to the invention is a good donor or acceptor of hydrogen bonds, selected from among the fatty alcohols, the fatty acids, the diols, the esters, the ethers, the pyrrolidones, the ketones, the sulfoxides, and their mixtures. The polar additive according to the invention is preferably selected from among octyldodecanol, hexyldecanol, octyldecanol, undecylpentadecanol, hexylene glycol, 1,2-hexanediol, propylene glycol, glycerine, oleic alcohol, phenylethyl alcohol, ricin oil, ethyl myristate, isopropyl palmitate, Finsolv, diisopropyl sebacate, diisopropyl adipate, propylene glycol dicaprate/dicaprylate and their mixtures.

The polar additive is advantageously present in the composition at a concentration of 1 to 40% by weight, preferably from 5 to 30% by weight, and more preferably from 8 to 20% by weight with respect to the total weight of the composition.

In general, the quantity of polar additive increases with the quantity of organo-gelator and the nature (apolar or weakly polar) of the liquid fatty phase; the quantity of additive must be sufficient to impart a thixotropic character to the composition and not totally to solubilize the organo-gelator at ambient temperature.

Organo-gelator

According to the invention, the composition comprises one or more molecular organo-gelators.

The molecules of organo-gelators are able to establish physical interactions between themselves leading to an auto-aggregation of the molecules with formation of a supra-molecular 3D network which is responsible for the gelation of the liquid fatty phase. The network may result from the formation of a network of fibrils (due to the stacking or aggregation of the molecules of the organo-gelator), immobilizing the molecules of the liquid fatty phase. Depending on the nature of the organo-gelator, the interconnected fibrils have variable dimensions ranging up to pm, even several pm. These fibrils may sometimes associate to form ribbons or columns.

The aptitude to form this network of fibrils, and thus gelation, depends on the nature (or chemical class) of the organo-gelator, on the nature of the substituents attached to its molecules for a given chemical class and on the nature of the liquid fatty phase.

The physical interactions are varied but exclude co-crystallization. These physical interactions are in particular interactions of the type auto-complementary hydrogen interactions, π interactions between unsaturated rings, dipolar interactions, co-ordination bonds with organometallic derivatives and their combinations. The establishment of these interactions is often favoured by the architecture of the molecule: rings, unsaturations, presence of asymmetric carbons. In general, each molecule of an organo-gelator may establish several types of physical interactions with a neighbouring molecule. The molecules of the organo-gelators according to the invention also advantageously contain at least one group able to establish hydrogen bonds and preferably at least two groups able to form hydrogen bonds, at least one aromatic ring and preferably at least two aromatic rings, at least one or more ethylenic unsaturations and/or at least one or more asymmetric carbons. The groups able to form hydrogen bonds are preferably selected from among the hydroxy, carbonyl, amine, carboxylic acid, amide, urea, benzyl groups and their combinations.

The organo-gelator or organo-gelators according to the invention are soluble in the liquid fatty phase after heating until a transparent homogenous liquid phase is obtained. They may be solid or liquid at ambient temperature and atmospheric pressure.

The organo-gelator or organo-gelators which may be used in the invention are especially those described in the document “Specialist Surfactants”, edited by D. Robb of 1997, p. 209-263, chapter 8 by P. Terech, the French patent applications (not yet published) 9909178, 0009317. They are most particularly the hydroxylated carboxylic fatty acids with a linear or branched aliphatic carbon chain containing in particular at least 8 carbon atoms and preferably at least 12 carbon atoms such as 12-hydroxystearic acid or 12-hydroxyoleic acid and their salts with alkali metals (Li, Na or K in particular) or alkaline earth metals (Mg for example); the amides of carboxylic acids, in particular tricarboxylic such as the cyclohexane tricarboxamides resulting from the reaction of cyclohexane tricarboxylic acid and a lauryl amine (see the French patent application n°9909178), these amides corresponding particularly to the formula (I) below; ureido derivatives such as the derivatives of 1,2-bis(ureido)benzene and trans-1,2-bis (ureido)cyclohexane, and in particular those described in the article by R. M. Kellog, B. L. Feringa et al. “Cyclic Bis-Urea Compounds as gelators for Organic Solvents” (Chem. Eur. J. 1999. 5. N°3); the amides or esters of amino acids such as the esters of alanine and the amides of valine, and in particular those described in the book “Specialist Surfactants”; the N-Acyl Aminoacids and their derivatives, and particularly the amides of N-acylamino acids such as the diamides resulting from the action of an N-acylamino acid with amines containing from 1 to 22 carbon atoms, such as for example those described in the international patent application WO-93/23008 and in particular the amides of N-acylglutamic acid where the acyl group represents a C₈ to C₂₂ alkyl chain; the diamides having from 1 to 22 carbon atoms, and preferably 6 to 18 atoms, the hydrocarbon chains being optionally substituted by ester, urea, fluoro groups (see French patent application n°0009317), these diamides are in particular those of formula (II) below; the amines or amides of steroids and particularly of deoxycholic, cholic, apocholic, lithocholic acids and their salts such as D-17,17-dipropyl-17a-aza-5α-homoandrostan-3ol or D-17,17-dipropyl-17a-aza-5α-homoandrostan-3β-ol 17a-oxy; compounds with several aromatic rings and particularly anthryl derivatives containing at least 2 alkyl chains having from 8 to 30 carbon atoms such as 2,3-bis-n-decyloxyanthracene, 2,3-bis-n-decyloxyanthraquinone or containing a steroid group such as cholesteryl 4-(2-anthryloxy)butanoate or cholesteryl anthraquinone-2-carboxylate and their derivatives; the azobenzene steroids such as those described in the book “Specialist Surfactants” organometallic compounds such as mononuclear copper β-diketonate (the complex of copper octa-substituted with bis(3,4 nonyloxy benzoyl) methanes), the binuclear copper tetracarboxylates or the complexes of Zn (II) with trisubstituted (para-carboxyphenyl) porphyrin; the surface-active agents in the form of salts containing at least two linear or branched alkyl chains and in particular the alkylphosphates of alkali metals or aluminium containing two alkyl chains having from 8 to 30 carbon atoms such as the aluminium salt of dihexadecyl phosphate (C₁₆) or di(2-ethyl hexyl) phosphoric acid and its alkali metal salts (Na), di(2-ethyl hexyl) sulfosuccinic acid and its alkali metal salts (Na); the benzylidene sorbitols or alditols and derivatives such as 1,3: 2,4-di-o-benzylidene-D-sorbitol, and their mixtures.

The preferred compounds are the amides of amino acids and more particularly the N-acylamino acids and their derivatives, the cyclohexane tricarboxamides described by the formula (I), the diamides described by the formula (II) and their mixtures.

Organo-gelator of Formula (I)

According to the invention, the organo-gelator may be a compound of formula (I) below:

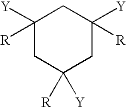

in which

R independently represents a hydrogen atom or a linear or branched saturated hydrocarbon chain, having 1 to 6 carbon atoms, particularly 1 to 4 carbon atoms;

Y represents a group chosen from among the following groups: —CO—S—R′; —CO—NHR′; —NH—COR′ and —S—COR′; in which R′ independently represents

a hydrogen atom,

an aryl group,

an aralkyl group, in other words aryl substituted by a linear or branched saturated hydrocarbon chain, containing 1 to 22 carbon atoms, particularly 10-18 carbon atoms; or

a linear, branched or cyclic saturated hydrocarbon chain, containing 1 to 22 carbon atoms, particularly 10-18 carbon atoms, optionally substituted by one or more groups chosen from among aryl, ester, amide, urethane groups; and/or optionally containing one or more heteroatoms chosen from among O, S and N; and/or optionally substituted by one or more fluorine atoms and/or hydroxy groups.

R preferably represents a hydrogen atom.

Y preferably represents a —CO—NHR′ or —NH—COR′ group.

R′ preferably represents an aryl group, an aralkyl group in which the linear or branched alkyl chain 12-16 carbon atoms; or a linear or branched C₁₂-C₁₈ alkyl chain.

Even more preferably, Y represents a —CO—NHR′ group in which R′ represents an aryl group substituted by a linear or branched C₁₂-C₁₆ alkyl chain; or R′ represents an unsubstituted linear or branched C₁₂-C₁₈ alkyl chain.

The three substituents represented by Y may be, in the compounds of formula (I), in a cis-cis, cis-trans or trans-trans conformation, with respect to each other. In particular, at least one of these substituents may be in an equatorial position on the cyclohexane ring. All the Y substituents are preferably in equatorial positions.

The compounds of formula (I) which may be used as organo-gelator, alone or in a mixture, in the composition of the invention, include:

cis-1,3,5-tris(dodecylaminocarbonyl)cyclohexane,

cis-1,3,5-tris(octadecylaminocarbonyl)cyclohexane,

cis-1,3,5-tris[N-(3,7-dimethyloctyl)-aminocarbonyl]cyclohexane,

trans-1,3,5-trimethyl-1,3,5-tris(dodecylaminocarbonyl)-cyclohexane, and

trans-1,3,5-trimethyl-1,3,5-tris(octadecylaminocarbonyl)-cyclohexane.

The compounds of formula (I) are well known to a person skilled in the art and may be prepared by the usual procedures.

Organo-gelator of Formula (II)

According to the invention, the organo-gelator may be a compound of formula (II) below

R—CO—NH—A—NH—CO—R′

in which

R and R′, which may be identical or different, represent a hydrogen atom or a saturated or unsaturated, linear, branched or cyclic hydrocarbon chain, containing 1 to 22 carbon atoms, particularly 6-18 carbon atoms, optionally substituted by one or more groups chosen from among aryl (—C₆H₅), ester (—COOR″ with R″ having 2 to 12 carbon atoms), amide (—CONHR″ with R″ having 2 to 12 carbon atoms), urethane (—OCONHR″ with R″ having 2 to 12 carbon atoms), urea (—NHCONHR″ with R″ having 2 to 12 carbon atoms); and/or optionally containing 1 to 3 heteroatoms chosen from among O, S and N; and/or optionally substituted by 1 to 4 halogen atoms, particularly fluorine and/or by 1 to 3 hydroxy groups, on the condition that R and/or R′ are different from hydrogen, and

A represents a saturated or unsaturated, linear, branched or cyclic hydrocarbon chain, containing 1 to 18 carbon atoms, particularly 2 to 12 carbon atoms, optionally substituted by one or more groups chosen from aryl (—C₆H₅), ester (—COOR″ with R″ having 2 to 12 carbon atoms), amide (—CONHR″ with R″ having 2 to 12 carbon atoms), urethane (—OCONHR″ with R″ having 2 to 12 carbon atoms), urea (—NHCONHR″ with R″ having 2 to 12 carbon atoms); and/or optionally containing 1 to 3 heteroatoms chosen from among O, S and N; and/or optionally substituted by 1 to 4 halogen atoms, particularly fluorine and/or by 1 to 3 hydroxy groups.

By unsaturated hydrocarbon chain according to formula (II), should be understood a chain containing at least one double bond c═c or at least one triple bond c≡c, said chain may additionally be optionally substituted by one or more groups chosen from among the aryl, ester, amide, urethane, urea groups and/or optionally containing 1 to 3 heteroatoms chosen from among O, S and N; and/or optionally substituted by one or more fluorine atoms and/or hydroxy groups.

By hydrocarbon chain according to formula (II) containing an atom of oxygen, sulfur or nitrogen, should be particularly understood a hydrocarbon chain containing a carbonyl (—C═O), amine (—NH2 or —NH—), thiol (—SH), thioether or ether group.

The compounds preferably correspond to formula (II) in which:

1/

A represents a hydrocarbon ring, saturated or unsaturated but not aromatic, optionally branched, having 4 to 12 carbon atoms, particularly 5 to 7 carbon atoms, optionally substituted by the substituents listed above and/or optionally containing 1 or more heteroatoms and/or optionally substituted by 1 or more halogen atoms and/or hydroxy groups

R and R′, which may be identical or different, represent a hydrogen atom or a saturated or unsaturated, linear, branched or cyclic hydrocarbon chain, preferably saturated linear, containing 10 to 16 carbon atoms, particularly 12 to 14 carbon atoms; or

2/

A represents a saturated, linear or branched hydrocarbon chain, having 2 to 18 carbon atoms, particularly 3 to 12 atoms, optionally substituted by the substituents listed above, and/or optionally containing 1 or more heteroatoms and/or optionally substituted by 1 or more halogen atoms and/or hydroxy groups;

R and R′, which may be identical or different, represent a hydrogen atom or a saturated or unsaturated, linear, branched or cyclic hydrocarbon chain, preferably saturated linear, containing 10 to 20 carbon atoms, particularly 11 to 18 carbon atoms, preferably 11 to 13 carbon atoms, and even more preferably 11 carbon atoms, on the condition that R and R′ are different from hydrogen; or

3/

A represents an aryl or aralkyl ring having 4 to 12 carbon atoms, particularly 5 to 8 carbon atoms, optionally substituted by the substituents listed above, and/or optionally containing 1 or more heteroatoms and/or optionally substituted by 1 or more halogen atoms and/or hydroxy groups;

R and R′, which may be identical or different, represent a hydrogen atom or a saturated or unsaturated, linear, branched or cyclic hydrocarbon chain, preferably saturated linear, containing 6 to 18 carbon atoms, particularly 10 to 16 carbon atoms, preferably 11 to 13 carbon atoms, and even more preferably 11 carbon atoms, on the condition that R and R′ are different from hydrogen.

The radical A may in particular represent a divalent radical of the cyclohexylene type (in particular 1,2-cyclohexylene, 1,3-cyclohexylene, 1,4-cyclohexylene; preferably 1,2-cyclohexylene), ethylene, propylene, isopropylene, butylene, isobutylene, pentylene, hexylene, dodecylene, dodecanylene, benzylene, phenylene, methyl-phenylene, bis-phenylene or napththalene, A may preferably be a divalent radical of the cyclohexylene, ethylene, propylene, isopropylene, dodecylene, methyl-phenylene type.

The radicals R and R′ may independently be selected from among the radicals pentyl, hexyl, decyl, undecyl, dodecyl, pentadecyl, hexadecyl, heptadecyl, octadecyl, 3-dodecyloxyproprionyl, 3-octadecyloxyproprionyl, 3-dodecyloxypentyl, 3-octadecyloxypentyl, 11-hydroxyheptadecyl. R and R′ may preferably be selected independently from among the radicals decyl, undecyl, dodecyl.

R and R′ are advantageously identical.

When the radical A is cyclic, the radicals R—CO—NH— and R′—CO—NH— may be in the ortho, meta or para position. In addition, they may be in the cis or trans position with respect to each other.

The compounds of formula (II) are preferably selected from among the compounds corresponding to one of the following formulas:

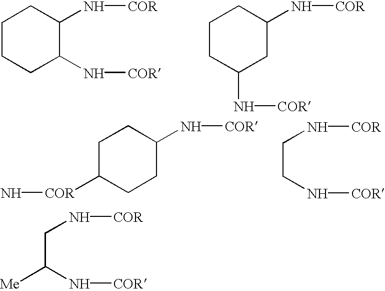

in which R and R′ have the same meanings as above.

The compounds able to be used as organo-gelators in the composition of the invention include:

N,N′-bis (dodecanoyl)-1,2-diaminocyclohexane, in particular in the trans form (compound of formula (I) with R=R′=n-C₁₁H₂₃ and A=divalent 1,2-cyclohexylene radical, also named (2-dodecanoylamino-cyclohexyl)-amide dodecanoic acid). This compound is particularly described in Hanabusa, K. Angew. Chem., 108, 1997, 17, pages 2086-2088.

N,N′-bis (dodecanoyl)-1,3-diaminocyclohexane, in particular in the trans form (compound of formula (I) with R=R′=n-C₁₁H₂₃ and A=divalent 1,3-cyclohexylene radical, also named (3-dodecanoylamino-cyclohexyl)-amide dodecanoic acid),

N,N′-bis (dodecanoyl)-1,4-diaminocyclohexane, , in particular in the trans form (compound of formula (I) with R=R′=n-C₁₁H₂₃ and A=divalent 1,4-cyclohexylene radical, also named (4-dodecanoylamino-cyclohexyl)-amide dodecanoic acid),

N,N′-bis (dodecanoyl)-1,2-ethylenediamine (compound of formula (I) with R=R′=n-C₁₁H₂₃ and A=divalent 1,2-ethylene radical, also named (2-dodecanoylamino-ethyl)-amide dodecanoic acid),

N,N′-bis (dodecanoyl)-1-methyl-1,2-ethylenediamine (compound of formula (I) with R=R′=n-C₁₁H₂₃ and A=divalent 1-methyl-1,2-ethylene radical, also named (2-dodecanoylamino-2-methylethyl)-amide dodecanoic acid),

N,N′-bis (dodecanoyl)-1,3-diaminopropane (compound of formula (I) with R=R′=n-C₁₁H₂₃ and A=divalent 1,3-propylene radical, also named (2-dodecanoylamino-propyl)-amide dodecanoic acid),

N,N′-bis (dodecanoyl)-1,12-diaminododecane (compound of formula (I) with R=R′=n-C₁₁H₂₃ and A=divalent 1,12-dodecylene radical, also named (2-dodecanoylamino-dodecyl)-amide dodecanoic acid),

N,N′-bis (dodecanoyl)-3,4-diaminotoluene (compound of formula (I) with R=R′=n-C₁₁H₂₃ and A=divalent 1-methyl-3,4-phenylene radical, also named (2-dodecanoylamino-4-methyl-phenyl)-amide dodecanoic acid),

their mixtures.

The compounds of formula (II) may be prepared according to methods well known to a person skilled in the art and be used, alone or in mixtures, in the composition of the invention.

General Method of Preparation of Compounds of Formula (II)

The diamine and two equivalents of triethylamine are dissolved in 50 ml of tetrahydrofuran. Two equivalents of acyl chloride in solution in THF are added, the reaction mixture is heated to reflux temperature of the THF, and the disappearance of the acyl chloride is monitored by IR spectroscopy (conventionally, two hours). The precipitate is removed by filtration, and the organic phase is concentrated and a liquid-liquid extraction is performed of the solid compound obtained. The organic phase is then dried, then concentrated and the solid product obtained is recrystallized.

The N-acylamino Acids and Their Derivatives

Examples of N-Acyl-amino acids and their derivatives are described in the U.S. Pat. No. 3,969,087.

These N-Acyl-amino acids and their derivatives are compounds of the general formula (III):

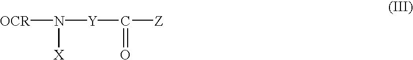

in which:

RCO represents an acyl group, containing from 2 to 30 carbon atoms, or an acyl aralkyl group,

Z represents a hydroxy group, NH2, —OR₁, —NRH₂,

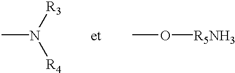

where R₁ to R₅ independently represent hydrocarbon chains, containing from 1 to 30 carbon atoms, or aralkyl groups,

X represents a hydrogen atom or a methyl group, and

Y is a hydrocarbon chain or a benzyl or phenyl group, or —CH₂OH,

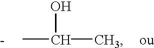

—CH₂SH, —CH₂CH₂S—CH₃, or —(CH₂)ₚ—COZ where p is a positive integer chosen from the numbers 1 and 2, and Z is defined as above, or —(CH2)ₙ— where n is a positive integer chosen between 1 and 6 on the condition that Z represents OR₁, Y represents

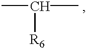

and at least one of the R₁ to R₅ groups is a hydrocarbon chain.

RCO is preferably an acyl group containing from 12 to 30 carbon atoms, X is a hydrogen atom and Y is

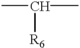

where R₆ represents —(CH₂)ₚ—COZ where Z is NHR₂.

The particularly preferred organo-gelators are the amides of the N-Acylamino acids of general formula (IV):

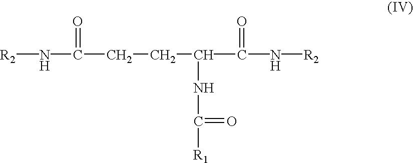

where R₁ is a hydrocarbon chain containing from 12 to 22 carbon atoms and R₂ is a hydrocarbon chain containing from 2 to 22 carbon atoms.

The amino acids may be D or L isomers, but the L isomers are preferred.

Examples of compounds having the general formula (IV) include the dibutylamide of n-lauroyl-L-glutamic acid, the diheptylamide of n-stearoyl-L-glutamic acid, the diethylamide of N-Lauroyl-L-glutamic acid, the dioctylamide of N-Lauroyl-L-glutamic acid, the didecylamide of N-Lauroyl-L-glutamic acid, N-Lauroyl-L-glutamic acid, the ditetradecylamide, etc.

The preferred organo-gelator of formula (IV) is the dibutylamide of N-Lauroyl-L-glutamic acid, commercially available from the Company Ajinomoto under the name GP-1 or from the Company CLARIANT under the name LGB.

The esters of the N-Acylamino acids of general formula (IV) are also preferred organo-gelators according to the invention.

Examples of such esters include the esters of N-lauroyl-α-alanine, of N-lauroyl-valine, of N-lauroyl-L-glutamic acid, of N^{α}N^{δ}-dicaproyl-ornithine, of N^{α}N^{δ}-dicaproy-ornithine-octyl, decyl, lauryl and stearyl, and of N^{α}N^{δ}-dicaproyllysine-octyl, decyl, and lauryl.

The composition advantageously contains from 0.1 to 60 % by weight of organo-gelator, preferably from 1 to 30 % by weight, and more preferably from 2 to 20 % by weight with respect to the total weight of the composition.

Fatty Phase

The liquid fatty phase according to the invention may contain one or more apolar or weakly polar liquid oils.

The apolar oils according to the invention are in particular the silicone oils such as the polydimethylsiloxanes (PDMS), volatile or not, linear or cyclic, liquid at ambient temperature; the polydimethylsiloxanes containing alkyl or alkoxy groups, within or at the end of the silicone chain, these groups each having from 2 to 24 carbon atoms; the phenylated silicones such as the phenyl trimethicones, the phenyl dimethicones, the phenyl trimethylsiloxy diphenylsiloxanes, the diphenyl dimethicones, the diphenyl methyldiphenyl trisiloxanes, the 2-phenylethyl trimethylsiloxysilicates; the linear or branched hydrocarbons of synthetic or natural origin, volatile or not, such as the paraffin oils, volatile (isoparaffins or isododecane) or non-volatile, and their derivatives, Vaseline, liquid lanolin, the polydecenes, hydrogenated polyisobutene such as Parleam (from NIPPON Gel Fats), squalane; the fluorinated, especially perfluorinated oils; the fluorinated silicones; and their mixtures.

The apolar oils according to the invention are preferably the oils or mixtures of oils of natural or synthetic origin, selected in particular from the hydrocarbons, especially the alkanes such as Parleam oil, the isoparaffins such as isododecane and squalane and their mixtures.

By oils of the hydrocarbon type, should be understood, in the context of the invention, oils containing hydrogen and carbon atoms only.

The weakly polar oils according to the invention, having a polarity parameter δₐ less than 7.0 (J/cm³)^{½}, are selected from among the esters of C₈-C₇₀ fatty acids or alcohols such as ethyl hexyl palmitate, tridecyl trimellitate, isopropyl myristate, myristyl myristate, octyl-dodecyl myristate, butyl stearate, isopropyl stearate, ethyl-hexyl stearate, octyldodecyl stearate, stearyl heptanoate, stearyl caprylate, dilsopropyl adipate, isostearyl neopentanoate, isopropyl isostearate, isooctadecyl isononanoate, isononyl isononanoate.

The fatty phase contains:

either at least one apolar oil present in the composition at a concentration of from 0.2 to 98.9% by weight, preferably at a concentration of from 1 to 80% by weight, and more preferably from 5 to 60% by weight with respect to the weight of the fatty composition,

or at least one weakly polar oil having a polarity parameter δₐ less than 7.0 (J/cm³)^{½}, present in the composition at a concentration of from 0.2 to 98.9% by weight, preferably at a concentration of from 1 to 80% by weight, and more preferably from 5 to 60% by weight with respect to the weight of the fatty phase.

The total liquid fatty phase represents, in practice, from 0.2 to 98.9% by weight, preferably from 10 to 80% by weight, and more preferably from 5 to 60% by weight of the total weight of the composition. It may contain apolar oils only, or weakly polar oils, or a mixture of these oils.

The composition according to the invention may, in addition, contain one or more polar oils, in other words oils having a polarity parameter δₐ equal to or greater than 7.0 (J/cm³)^{½}, constituting in particular the polar additive, selected from among octyldodecanol, hexyldecanol, octyidecanol, undecylpentadecanol, hexylene glycol, 1,2-hexanediol, propylene glycol, glycerine, oleic alcohol, phenylethyl alcohol, ricin oil, ethyl myristate, isopropyl palmitate, Finsolv, dilsopropyl sebacate, diisopropyl adipate, propylene glycol dicaprate/dicaprylate and their mixtures.

A further object of the invention is a cosmetic composition comprising at least one liquid fatty phase containing at least one apolar or weakly polar hydrocarbon oil, having a polarity parameter δₐ less than 7.0 (J/cm³)^{½}, structured by at least one molecular organo-gelator of the liquid fatty phase to the exclusion of any molecular gelling agent with average molecular weight of from 1000 to 100 000 and comprising a polymeric skeleton having urea, urethane, thiourea, thiourethane and amide groups, and at least one polar additive having a polarity parameter δₐ equal to or greater than 7.0 (J/cm³)^{½}, the fatty phase, the organo-gelator, and the additive forming an physiologically acceptable medium.

The polar additive of the composition according to the invention is such as defined above.

The molecular organo-gelator of the composition according to the invention is such as defined above and is preferably selected from the N-acylamino acids, the amides of N-acylamino acids, the cyclohexane tricarboxamides, the cyclohexane diamides, and their mixtures.

The apolar hydrocarbon oils of the composition according to the invention are advantageously selected from among the hydrogenated polyisobutenes such as Parleam (available from Nippon Gel Fats), squalane, the aliphatic hydrocarbons, particularly C₆-C₄₀, and their mixtures.

Preferred apolar oils according to the invention include the silicones such as the phenyltrimethicones and cyclopentadimethylsiloxane.

The preferred weakly polar hydrocarbon oils of the composition according to the invention are the oils having a polarity parameter δₐ less than 7.0 (J/cm³)^{½}, selected from among ethyl hexyl palmitate, tridecyl trimellitate, isononyl isononanoate, isopropyl myristate and their mixtures. The constituents of the formula are those described above.

The composition, according to the invention, shows a particular texture and rheology.

The addition of the polar additive does not in fact change the gelling capacity of the organo-gelator in the apolar or weakly polar oils according to the invention.

However, the progressive addition of the polar additive induces a reinforcement of the gelation which results in an increase of the consistency (viscosity) of the compositions up to a maximum concentration Cₘₐₓ in polar additive. Beyond Cₘₐₓ, the consistency and the solid character of the compositions decreases. In fact, the compatibility of the organo-gelator with the fatty phase is strongly increased, a partial dissolution of the organo-gelator occurs, and as a result the composition is less consistent and less solid.

By “solid” should be understood, in the context of the invention, a composition which does not flow under its own weight at ambient temperature. By “increase of the compatibility”, should be understood a solubilization of the organo-gelator in the liquid fatty phase at a lower temperature.

In addition, thanks to the polar additive, the properties under shearing of the composition of the invention are improved. In particular, a better fluidization of the composition (slow and regular reduction of the apparent viscosity under the effect of shearing) is observed, and especially an improved spreading onto keratin-containing material. The phenomenon of breaking-up, or caking, of the deposit of the composition, which occurs in the absence of the polar additive, is reduced; the textures obtained after strong shearing are smoother and more homogenous. In addition, the crumbling qualities of the compositions are significantly improved with the addition of an effective quantity of the polar additive.

Moreover, the aptitudes to restructuring of the compositions after spreading vary according to the nature of the oil and the quantity of polar additive added. The property of the composition to recover its viscosity and consistency are considerably improved by the addition of the polar additive in the combination {organo-gelator+oil}. As the concentration in polar additive increases, the property of recovery of viscosity and consistency are improved, up to Cₘₐₓ where the composition containing the polar additive has a level of consistency after shearing 10 times larger than that of the composition not containing the polar additive. The intensity of this phenomenon (properties of recovery of restructuring) is slightly less marked in Parleam than in 2-ethyl hexyl palmitate and still less in tridecyl trimellitate, where the kinetics of recovery are slower, although still satisfactory.

The composition of the invention may additionally contain any complementary additive normally used in the field concerned and in particular the cosmetic or dermatological fields, especially selected from the antioxidants, essential oils, preservatives (BHT), perfumes, fillers (Polytrap® from Dow Corning), neutralizing agents, gums, polymers which are liposoluble or dispersible in the medium other than the polyureas, polyurethanes, polythioureas and polythiourethanes, cosmetic or dermatological active agents such as emollients, hydrating agents, vitamins, essential fatty acids, dispersants such as poly(12-hydroxystearique) acid and their mixtures.

Each additive may be present in the composition at a concentration of from 0 to 20% (particularly from 0.01 to 20 %) of the total weight of the composition, and preferably from 0.01 to 10% (if present).

The composition according to the invention may additionally contain at least one fatty compound which is pasty at ambient temperature. By “pasty fatty body”, in the context of the invention, should be understood a fatty body having a melting point of from 20 to 55 ° C., preferably from 25 to 45° C., andlor a viscosity at 40° C. of from 0.1 to 40 Pa·s (1 to 400 poises), preferably 0.5 to 25 Pa·s, measured with a Contraves TV or Rheomat 80, fitted with a moving piece turning at 200 r.p.m. A skilled person may select the moving piece to measure the viscosity, from the pieces MS-r3 and MS-r4, based on his general knowledge, so as to perform the measurement on the pasty compound tested.

The pasty compounds able to be used in the composition according to the invention include the lanolins and lanolin derivatives such as the acetylated lanolins or the oxypropylated lanolins or isopropyl lanolin, having a viscosity of 18 to 21 Pa·s, preferably 19 to 20.5 Pa·s, and/or a melting point of 30 to 55° C., and their mixtures. Other possible compounds are the esters of fatty acids or alcohols, particularly those having 20 to 65 carbon atoms (melting point of the order of 20 to 35° C. and/or viscosity at 40° C. of from 0.1 to 40 Pa·s) such as tri-isostearyl or cetyl citrate; arachidyl propionate; vinyl polylaurate; the esters of cholesterol such as the triglycerides of plant origin such as the hydrogenated plant oils (hydrogenated ricin oil), the viscous polyesters such as poly(12-hydroxystearic) acid; the polydimethylsiloxanes (PDMS) having side chains of the alkyl or alkoxy type having from 8 to 24 carbon atoms, and a melting point of 20-55° C., such as the stearyl dimethicones (especially DC2503 and DC25514 from Dow Corning); and their mixtures.

The pasty fatty body or bodies may be present at a concentration of from 0 to 60% by weight, with respect to the total weight of the composition, preferably from 0.1 to 45% by weight, and even more preferably from 2 to 30% by weight, in the composition, if they are present.

The composition of the invention may additionally contain as a complementary additive an aqueous phase containing water, optionally thickened or gelified by a thickener or gelling agent of the aqueous phase, and optionally compounds miscible with water. The aqueous phase may represent from 0 to 60% by weight, and particularly from 0.5 to 60% by weight of the total weight of the composition.

The person skilled in the art will obviously take care that the choice and/or quantity of any complementary additives is such that the advantageous properties of the composition according to the invention are not, or are not significantly, modified by any adjunction envisaged.

The composition according to the invention may be in the form of a care composition, coloured or not, for keratin-containing materials such as the skin, lips, hair and/or nails, or in the form of a personal hygiene composition, especially in the form of a deodorant or make-up removal product in the form of a stick, or in the form of a lip make-up composition, called “top coat”, for application to the lips in two layers to prevent its migration. It may in particular be used as a basis for a care composition for the skin, hair, nails or lips (lip balm, protecting the lips from cold and/or sun and/or wind, care cream for the skin, nails or hair).

The composition advantageously contains at least one colorant.

The colorant according to the invention may be selected from the lipophilic colorants, the hydrophilic colorants, the pigments and the nacres normally used in cosmetic or dermatological compositions, and their mixtures. This colorant is generally present in a concentration of 0.01 to 90% of the total weight of the composition, preferably from 0.5 to 60% and more preferably from 2 to 30%, if it is present. In the case of a composition in the form of a free or compacted powder, the quantity of colorant in the form of insoluble solid particles in the medium (nacres and/or pigments) may be up to 90% of the total weight of the composition, compared to 60% for the other cosmetic forms.

The liposoluble colorants are, for example, Sudan red, D&C Red 17, D&C Green 6, β-carotene, soya oil, Sudan brown, D&C Yellow 11, D&C Violet 2, D&C orange 5, quinoline yellow, annatto. They may represent from 0.1 to 20% of the weight of the composition and preferably from 0.1 to 6% (if present). The water-soluble colorants are for example beetroot juice, methylene blue, and may represent up to 6 % of the total weight of the composition.

The pigments may be white or coloured, inorganic and/or organic, coated or not. The inorganic pigments include titanium dioxide, optionally surface-treated, zirconium, zinc or cerium oxides, and the iron and chromium oxides, manganese violet, ultramarine blue, chromium hydrate and ferric blue. The organic pigments include carbon black, the pigments of type D&C, and the lakes based on cochineal carmine, barium, strontium, calcium, aluminium. The pigments may represent from 0.1 to 50% of the total weight of the composition, if present.

The nacre pigments may be selected from the white nacre pigments, such as mica coated with titanium or bismuth oxychloride, the coloured nacre pigments such as titanium mica with iron oxides, titanium mica with in particular ferric blue or chromium oxide, titanium mica with an organic pigment of the type mentioned above, and the nacre pigments based on bismuth oxychloride, or the interferential or gogniochromatic pigments. They may represent from 0.1 to 30% of the total weight of the composition and preferably from 0.1 to 20%, if present, and for example from 1 to 15%.

The composition of the invention may also be in the form of a make-up product, in particular coloured, for the skin, especially a foundation cream, optionally with skin-care properties, a blush, eye shadow, concealer, eye-liner; a make-up product for the lips, such as a lipstick, optionally with care properties, a lip gloss, a lip pencil; a make-up product for the nails or hair, such as the eyelashes particularly in the form of a mascara block, or the eyebrows and the hair in the form of a pencil.

The composition of the invention must obviously be cosmetically or dermatologically acceptable, in other words contain a non-toxic physiologically acceptable medium which may be applied to the skin, hair, nails or lips of humans.

By cosmetically acceptable, in the context of the invention, should be understood a composition with an agreeable appearance, smell, taste and feel.

The invention is illustrated in further detail in the following examples. The quantities are given in percentages by weight.

### EXAMPLES

### Comparative examples E₁ to E₄

A first case of the control composition E₁ was prepared by mixing 2-ethylhexyl palmitate and LGB. The control composition E₁ did not contain an polar additive.

Three cases of the composition according to the invention E₂ to E₄ were also prepared by mixing 2-ethylhexyl palmitate, LGB and octyldodecanol as polar additive.

The compositions E₁ to E₄ are given in table 1 below.

TABLE 1

| | Octyldodecanol | | 2-ethylhexyl |
|---|---|---|---|
| Compositions | (polar additive) | LGB | palmitate |
| E₁ (control) | 0% | 2% | qsp 100% |
| E₂ | 5% | 2% | qsp 100% |
| E₃ | 10% | 2% | qsp 100% |
| E₄ | 20% | 2% | qsp 100% |

For all these cases of compositions E₁ to E₄, the mixture of 2-ethylhexyl palmitate, LGB and octyidodecanol (where appropriate) was effected by agitation at ambient temperature. The mixture was heated until a transparent homogenous fluid phase was obtained (the temperature necessary for this homogenization varied between 90 and 150° C. as a function of the proportions of octyidodecanol). The fluid and homogeneous mixture was allowed to cool to ambient temperature. Solid hard compositions were thus obtained, which did not collapse at ambient temperature.

The complex modulus G*=(G′²+G″²)^{½} which expresses the consistency of the products at 25° C., expressed in Pa, as well as the loss angle or phase difference δ which expresses the solid or viscous character of the product, were measured.

A Haake RS 150 (Rheo Company) rheometer with applied stresses, with a cone-plane measurement unit of diameter 35 mm and angle 2°, whose surfaces were sanded to avoid slipping problems, was used for measurements. All measurements were made at 25° C.

For the parameters obtained after shearing, each sample was subjected to a series of sinusoidal stresses of fixed frequency (1 Hz) and increasing amplitudes distributed logarithmically between the two limits at a level of 5 stresses per decade.

The sheared samples were subjected to a shearing speed of 1000 s⁻¹ for 30 s. The restructuring capacities of the samples were then determined by the monitoring over time of the parameters G* and δ defined above. For this, the samples were subjected to a series of sinusoidal stresses of fixed frequency (1 Hz) of constant amplitude located in the linear viscoelastic region.

The Theological parameters measured were as follows

the conservation modulus or elastic modulus G′,

the loss modulus or viscous modulus G″,

the complex modulus G*,

the loss angle or phase difference δ, with tan(δ)=G″/G′; the more elastic the material, the more δ approaches 0° and the more viscous, the more δ approaches 90°.

The parameters G* and δ before and after shearing are given in table 2.

TABLE 2

| | | δ after | | G* after | |
|---|---|---|---|---|---|
| | δ before | shearing and | G* (Pa) | shearing and t | |
| | shearing | 10 mn rest | before | mn rest (in °) | |
| Composition | (in °) | (in °) | shearing | t = 1 | t = 10 |
| E₁ | 7.5 | 9 | 1.3 × 10⁵ | 1080 | 2660 |
| E₂ | 7.7 | 5 | 1.7 × 10⁵ | 3580 | 20800 |
| E₃ | 7.6 | 3 | 1.5 × 10⁵ | 7200 | 29700 |
| E₄ | 7.7 | 5.6 | 1.4 × 10⁵ | 3970 | 24700 |

The compositions E₁ to E₄ were solid, with a firm consistency before spreading.

Moreover, there was a recovery of the consistency and improved hardness in the presence of octyidodecanol (E₂ to E₄) compared to the control composition E₁ without octyldodecanol.

In addition, the solid character as defined by the loss angle was maintained before and after shearing. The lower the value of δ, the more the composition had a solid character. The measurement differences shown in the table are not significant.

Sensory tests also showed that the compositions according to the invention (E₂ to E₄) were less migratory than the control composition E₁ not containing octyidodecanol.

### Example of Composition 1

Solid Foundation

| | |
|---|---|
| GP-1 from Ajinomoto | 2% |
| Octyldodecanol | 8% |
| Pigments (yellow and brown iron oxide) | 10% |
| Nylon ® powders | 3% |
| Perfume | 0.2% |
| BHT | 0.07% |
| 2-Ethylhexyl palmitate | qsp 100% |

1. Use of at least one polar additive having a polarity parameter δₐ equal to or greater than 7.0 (J/cm³)^{½}, in a cosmetic composition comprising a liquid fatty phase containing at least one apolar or weakly polar oil having a polarity parameter δₐ less than 7.0 (J/cm³)^{½}, structured by at least one molecular organo-gelator, to impart a thixotropic character to the composition.

2. Use of at least one polar additive having a polarity parameter δₐ equal to or greater than 7.0 (J/cm³)^{½}, in a cosmetic composition comprising a liquid fatty phase containing at least one apolar or weakly polar oil having a polarity parameter δₐ less than 7.0 (J/cm³)^{½}, structured by at least one molecular organo-gelator, as an agent to reduce or eliminate the migration of the fatty phase.

3. Use of at least one polar additive having a polarity parameter δₐ equal to or greater than 7.0 (J/cm³)^{½}, in a cosmetic composition comprising a liquid fatty phase containing at least one apolar or weakly polar oil having a polarity parameter δₐ less than 7.0 (J/cm³)^{½}, structured by at least one molecular organo-gelator, as an agent to reduce or eliminate the formation of ridges on the eyelids.

4. Use of at least one polar additive having a polarity parameter δₐ equal to or greater than 7.0 (J/cm³)^{½}, in a cosmetic composition comprising a liquid fatty phase containing at least one apolar or weakly polar oil having a polarity parameter δₐ less than 7.0 (J/cm³)^{½}, structured by at least one molecular organo-gelator, to eliminate the tendency of the composition to break up, and/or encourage the spreading of the composition onto the skin and/or the formation of a smooth, homogenous, non-granulous and continuous deposit on keratin-containing materials.

5. Use according to any of claims 1 to 4, characterized in that the polar additive is a good donor or acceptor of hydrogen bonds, selected from among the fatty alcohols, the fatty acids, the diols, the esters, the ethers, the pyrrolidones, the ketones, the sulfoxides, and their mixtures.

6. Use according to claim 5, characterized in that the additive is selected from octyldodecanol, hexyldecanol, octyidecanol, undecylpentadecanol, hexylene glycol, 1,2-hexanediol, propylene glycol, glycerine, oleic alcohol, phenylethyl alcohol, ricin oil, ethyl myristate, isopropyl palmitate, Finsolv, diisopropyl sebacate, diisopropyl adipate, propylene glycol dicaprate/dicaprylate and their mixtures.

7. Use according to claim 5 or 6, characterized in that the polar additive is present in the composition at a concentration of 1 to 40% by weight, preferably from 5 to 30% by weight, and more preferably from 8 to 20% by weight with respect to the total weight of the composition

8. Use according to any of the preceding claims, characterized in that the molecular organo-gelator is an organo-gelator whose molecules contain at least one group able to establish hydrogen bonds and preferably at least two groups able to form hydrogen bonds, at least one aromatic ring and preferably at least two aromatic rings, at least one or more ethylenic unsaturations and/or at least one or more asymmetric carbons.

9. Use according to any of the preceding claims, characterized in that the molecular organo-gelator is selected from the hydroxylated carboxylic acids with a linear or branched aliphatic carbon chain containing in particular at least 8 carbon atoms; the amides of carboxylic acids; the amides or esters of amino acids; the amides of N-acylamino acids and particularly the diamines resulting from the action of an N-acylamino acid with amines containing from 1 to 22 carbon atoms; the diamides having from 1 to 22 carbon atoms, and preferably 6 to 18 atoms, the hydrocarbon chains being optionally substituted; the amines or amides of steroids and their salts; the azobenzene steroids; organometallic compounds; the surface-active agents in the form of salts containing at least two linear or branched alkyl chains having from 8 to 30 carbon atoms; the benzylidene sorbitols or alditols and derivatives; and their mixtures.

10. Use according to any of the preceding claims, characterized in that the molecular organo-gelator is selected from among the N-acylamino acids, the amides of N-acylamino acids, the cyclohexane tricarboxamides, the cyclohexane diamines and their mixtures.

11. Use according to any of the preceding claims, characterized in that the molecular organo-gelator represents from 0.1 to 60% by weight, preferably from 1 to 30% by weight, and more preferably from 2 to 20 % by weight of the total weight of the composition.

12. Use according to any of the preceding claims, characterized in that the apolar oil is a hydrocarbon oil selected from among parleam oil, squalane, the aliphatic hydrocarbons, and their mixtures.

13. Use according to any of claims 1 to 11, characterized in that the apolar oil is an apolar silicone oil selected from among the polydimethylsiloxanes (PDMS) and the phenylated silicones such as the phenyltrimethicones, the phenyidimethicones, the diphenyidimethicones, the diphenyl methyidiphenyl trisiloxanes and the 2-phenylethyl trimethylsiloxysilicates, and their mixtures.

14. Use according to any of claims 1 to 11, characterized in that the weakly polar oils, having a polarity parameter δₐ less than 7.0 (J/cm³)^{½}, are selected from among the esters of C₈-C₇₀ fatty acids or alcohols, such as ethyl hexyl palmitate and tridecyl tri mellitate.

15. Use according to any of the preceding claims, characterized in that the fatty phase contains:
either at least one apolar oil present in the composition at a concentration of from 0.2 to 98.9% by weight, preferably at a concentration of from 1 to 80% by weight, and more preferably from 5 to 60% by weight with respect to the weight of the fatty phase,
or at least one weakly polar oil, having a polarity parameter δₐ less than 7.0 (J/cm³)^{½}, present in the composition at a concentration of from 0.2 to 98.9% by weight, preferably at a concentration of from 1 to 80% by weight, and more preferably from 5 to 60% by weight with respect to the weight of the fatty phase.

16. Use according to any of the preceding claims, characterized in that the liquid fatty phase represents from 0.2 to 98.9% by weight, preferably from 10 to 80% by weight, and more preferably from 5 to 60% by weight of the total weight of the composition.

17. Use according to any of the preceding claims, characterized in that the liquid fatty phase also contains one or more polar oils, having a polarity parameter δₐ equal to or greater than 7.0 (J/cm³)^{½}, selected from among octyidodecanol, hexyldecanol, octyidecanol, undecylpentadecanol, hexylene glycol, 1,2-hexanediol, propylene glycol, glycerine, oleic alcohol, phenylethyl alcohol, ricin oil, ethyl myristate, isopropyl palmitate, Finsolv, diisopropyl sebacate, diisopropyl adipate, propylene glycol dicaprate/dicaprylate and their mixtures.

18. Cosmetic composition comprising at least one liquid fatty phase containing at least one apolar or weakly polar hydrocarbon oil, having a polarity parameter δₐ less than 7.0 (J/cm³)^{½}, structured by at least one molecular organo-gelator of said liquid fatty phase to the exclusion of any polymeric gelling agent of average molecular weight from 1000 to 100 000 and comprising a polymeric skeleton having urea, urethane, thiourea, thiourethane and/or amide groups, and at least one polar additive having a polarity parameter δₐ equal to or greater than 7.0 (J/cm³)^{½}, the fatty phase, the molecular organo-gelator, and the additive forming a physiologically acceptable medium.

19. Composition according to claim 18, characterized in that the additive is selected from among the fatty alcohols, the fatty acids, the diols, the esters, the ethers, the pyrrolidones, the ketones, the sulfoxides, and their mixtures.

20. Composition according to claim 19, characterized in that the additive is selected from among octyldodecanol, hexyldecanol, octyldecanol, undecylpentadecanol, hexylene glycol, 1,2-hexanediol, propylene glycol, glycerine, oleic alcohol, phenylethyl alcohol, ricin oil, ethyl myristate, isopropyl palmitate, Finsolv, diisopropyl sebacate, diisopropyl adipate, propylene glycol dicaprate/dicaprylate and their mixtures.

21. Composition according to any of claims 18 to 20, characterized in that the molecular organo-gelator is selected from among the hydroxylated fatty carboxylic acids with a linear or branched aliphatic carbon chain containing in particular at least 8 carbon atoms; the amides of carboxylic acids; the amides or esters of amino acids; the amides of N-acylamino acids and particularly the diamides resulting from the action of an N-acylamino acid with amines containing from 1 to 22 carbon atoms; the diamides having from 1 to 22 carbon atoms, and preferably 6 to 18 atoms, the hydrocarbon chains being optionally substituted; the amines or amides of steroids; the azobenzene steroids; organometallic compounds; the surface-active agents in the form of salts containing at least two linear or branched alkyl chains having from 8 to 30 carbon atoms; the benzylidene sorbitols or alditols and derivatives; and their mixtures.

22. Composition according to claim 21, characterized in that the molecular organo-gelator is selected from among the N-acylamino acids, the amides of N-acylamino acids, the cyclohexane tricarboxamides, the cyclohexane diamines and their mixtures.

23. Composition according to any of claims 18 to 22, characterized in that it constitutes a composition for care and/or make-up of keratin-containing materials.

24. Composition according to any of claims 18 to 23, characterized in that it contains at least one additive selected from among water, the antioxidants, essential oils, preservatives, perfumes, fillers, gums, fatty compounds which are pasty at ambient temperature, neutralizing agents, polymers which are liposoluble or dispersible in the medium, cosmetic or dermatological active agents, dispersants, and their mixtures, the additive being present at a concentration of from 0 to 20% by weight, particularly from 0.01 to 20 % by weight, and preferably from 0.01 to 10% by weight of the total weight of the composition.

25. Composition according to any of claims 18 to 24, characterized in that it is anhydrous and is in the form of a rigid gel, and particularly of an anhydrous stick.

26. Composition according to any of claims 18 to 24, characterized in that it is in the form of a deodorant product.

27. Composition according to any of claims 18 to 24, characterized in that it is in the form of a top coat.

28. Composition according to any of claims 18 to 27, characterized in that it contains at least one colorant selected from among the lipophilic colorants, the hydrophilic colorants, the pigments, the nacres and their mixtures, the colorant being present at a concentration of from 0.01 to 50% by weight, and preferably from 0.5 to 40% by weight with respect to the total weight of the composition.

29. Composition according to claim 28, characterized in that it is in the form of a lipstick.
